(19) United States
(12) Patent Application Publication
(10) Pub. No.: US 2017/0258774 A1
(43) Pub. Date: Sep. 14, 2017

(54) NOVEL OXIMES FOR REACTIVATING BUTYRYLCHOLINESTERASE

(71) Applicants: Janice E. Chambers, Starkville, MS (US); Howard W. Chambers, Starkville, MS (US); Edward C. Meek, Starkville, MS (US)

(72) Inventors: Janice E. Chambers, Starkville, MS (US); Howard W. Chambers, Starkville, MS (US); Edward C. Meek, Starkville, MS (US)

(73) Assignee: MISSISSIPPI STATE UNIVERSITY, Starkville, MS (US)

(21) Appl. No.: 15/458,957

(22) Filed: Mar. 14, 2017

Related U.S. Application Data
(60) Provisional application No. 62/307,810, filed on Mar. 14, 2016.

(51) Int. Cl. A61K 31/4425

(52) U.S. Cl. CPC A61K 31/4425

(57) ABSTRACT

Oxime molecules for reactivating butyrylcholinesterase (BChE) and methods for protection against and therapeutic treatment of the toxic effects of organophosphorus compounds (OP) cholinesterase inhibitors such as nerve agents and/or insecticides are provided. The oxime molecules can be administered to a subject in need thereof to treat or prevent toxic effects of OPs. The oxime molecules can allow for a dual reactivation treatment paradigm by reactivating both serum BChE and inactivated CNS AChE.

### CROSS-REFERENCE TO RELATED APPLICATIONS

This application claims priority to U.S. Provisional Application No. 62/307,810, to Janice E. Chambers et al. filed on Mar. 14, 2016, the contents of which are incorporated herein by reference in its entirety.

### STATEMENT OF GOVERNMENT SUPPORT

This invention was made with government support under grant U01 NS083430 awarded by the NIH-CounterACT. The government has certain rights in the invention.

### FIELD OF THE INVENTION

This invention relates to the field of nerve agent antidotes and protection against the toxic effects of organophosphorus (OP) cholinesterase inhibitors such as nerve agents and/or insecticides. More specifically, the invention relates to novel oximes that reactivate butyrylcholinesterase (BChE) and methods for protection against and therapeutic treatment of the toxic effects of such OP cholinesterase inhibitors.

### BACKGROUND OF THE INVENTION

Organophosphorus compounds (OPs), such as nerve agents and insecticides, pose a threat to military personnel and civilians due to their potential use in a direct fashion (terrorist or military attack) or indirect fashion (accidental poisoning). This was made evidently clear in recent years by the doomsday cult Aum Shinrikyo in 1995 and the current Syrian civil war. OPs inhibit serine hydrolases, such as acetylcholinesterase (AChE) and butyrylcholinesterase (BChE). Inhibition of AChE leads to buildup of acetylcholine, an excitatory neurotransmitter found throughout the body, causing hyper-excitation of cholinergic pathways which can lead to respiratory failure and death if left untreated.

Oxime reactivators are critical therapeutics to treat OPs and are used to reverse the inhibited enzyme, namely AChE. Antidotal treatment focuses on blocking muscarinic cholinergic receptors with atropine and restoration of the inhibited enzyme by use of an oxime reactivator, such as 2-PAM. 2-PAM only works in the peripheral nervous system, leaving the CNS vulnerable to OP toxicity, and is not effective against all OP compounds. Currently, there has yet to be an effective reactivator that has broad spectrum capabilities. Thus broad spectrum and CNS-protecting oximes are needed.

### SUMMARY OF THE INVENTION

The present invention provides novel oximes that reactivate the enzyme BChE, a blood enzyme that is inhibited by OPs but does not lead to toxicity. The new oximes can be used as BChE reactivators for antidotes against OP anticholinesterase toxicity and includes methods for protecting against and therapeutic treatment of the toxic effects of OP anticholinesterases such as nerve agents and/or insecticides.

### BRIEF DESCRIPTION OF THE DRAWINGS

Further advantages of the invention will become apparent by reference to the detailed description of preferred embodiments when considered in conjunction with the drawings which form a portion of the disclosure and wherein:

FIG. 1—Chemical structures of known (PRIOR ART) OP nerve agents, surrogates, and insecticidal oxons;

FIG. 2—BChE Reactivation following Paraoxon. Novel oximes exhibited a reactivation range of 33-72%. 2-PAM averaged 32%. Data are expressed as mean±SEM, n=3. *P<0.05;

FIG. 3—BChE Reactivation following PIMP (sarin surrogate). Novel oximes exhibited a reactivation range of 45-73%. 2-PAM averaged 46%. Data are expressed as mean±SEM, n=3. *P<0.05;

FIG. 4—BChE Reactivation following NEMP (VX surrogate). Novel oximes exhibited a reactivation range of 18-62%. 2-PAM averaged 8%. Data are expressed as mean±SEM, n=3. *P<0.05;

FIG. 5—BChE Reactivation following Phorate Oxon. Novel oximes exhibited a reactivation range of 0-24%. 2-PAM averaged 70%. Data are expressed as mean±SEM, n=3. *P<0.05;

FIG. 6—BChE Reactivation following NCMP (cyclosarin surrogate). Novel oximes exhibited a reactivation range of 0-21%. 2-PAM averaged 4%. Data are expressed as mean±SEM, n=3. *P<0.05;

FIG. 7—BChE Reactivation following DFP. Novel oximes exhibited a reactivation range of 0-12%. 2-PAM averaged 2%. Data are expressed as mean±SEM, n=3. *P<0.05;

FIG. 8—Human BChE reactivation after exposure to PIMP and NEMP. Novel oximes showed a reactivation range of 47-78% to PIMP and 27-70% to NEMP. 2-PAM averaged 58% and 31%. Data are represented as mean±SEM. n=3 *p<0.05;

FIG. 9—Human BChE reactivation after exposure to paraoxon and phorate oxon. Novel oximes showed a reactivation range of 9-66% to paraoxon and 26-83% to phorate oxon. 2-PAM averaged 17% and 36%. Data are represented as mean±SEM. n=3 *p<0.05;

FIG. 10—Human BChE Reactivation following NCMP. Novel oximes exhibited a reactivation range of 8-65%. 2-PAM averaged 22%. Data are represented as mean±SEM. n=3 *p<0.05;

FIG. 11—GP BChE reactivation after exposure to PIMP and NEMP. Novel oximes showed a reactivation range of 42-51% to PIMP and 17-31% to NEMP. 2-PAM averaged 57% and 38%. Data are represented as mean±SEM. n=3 *p<0.05;

FIG. 12—GP BChE reactivation after exposure to paraoxon and phorate oxon. Novel oximes showed a reactivation range of 6-17% to paraoxon and 4-10% to phorate oxon. 2-PAM averaged 7% and 2%. Data are represented as mean±SEM. n=3 *p<0.05;

FIG. 13—Rat BChE reactivation after exposure to PIMP and NEMP. Novel oximes showed a reactivation range of 45-77% to PIMP and 18-88% to NEMP. 2-PAM averaged 57% and 38%. Data are represented as mean±SEM. n=3 *p<0.05;

FIG. 14—Rat BChE Reactivation following paraoxon. Novel oximes exhibited a reactivation range of 33-95%. 2-PAM averaged 32%. Data are represented as mean±SEM. n=3 *p<0.05;

FIG. 15—Rat BChE reactivation after exposure to phorate oxon and phorate oxon sulfoxide. Novel oximes showed a reactivation range of 0-24% to phorate oxon and 25-70% to phorate oxon sulfoxide. 2-PAM averaged 65% and 78%. Data are represented as mean±SEM. n=3*p<0.05;

FIG. 16—Rat BChE reactivation after exposure to NCMP and DFP. Novel oximes showed a reactivation range of 0-21% to NCMP and 0-20% to DFP. 2-PAM averaged 4% and 2%. Data are represented as mean±SEM. n=3 *p<0.05.

### DETAILED DESCRIPTION

The present invention provides novel oximes that reactivate butyrylcholinesterase (BChE) and methods for protection against and therapeutic treatment of the toxic effects of OP cholinesterase inhibitors such as nerve agents and/or insecticides. The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

### Definitions

As used herein, the following terms and variations thereof have the meanings given below, unless a different meaning is clearly intended by the context in which such term is used.

“Acyl” refers to a group of the form RCO—, where R is an organic group. The term “aroyl” refers to the group —C(O)R, where R is aryl. Similar compound radicals involving a carbonyl group and other groups are defined by analogy. The term “aminocarbonyl” refers to the group —NHC(O)—. The term “oxycarbonyl” refers to the group —OC(O)—.

“Administering” can be effected or performed using any of the various methods and delivery systems known to those skilled in the art. The administering or an administration can be performed, for example, intravenously, intraperitoneal injection, orally, nasally, rectally, intravaginally, topically, via implant, transmucosally, transdermally, intramuscularly, and subcutaneously. Due to the ability of BChE in the presence of an effective amount of oximes taught herein to be continually reactivated, a slow release formulation of an administered dose may be especially advantageous for a subject exposed to or at risk of exposure to an OP, in addition to any needed immediate bolus given to the subject to effectuate a desired bioavailable concentration of the oxime(s).

“Alkyl” refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “heteroalkyl” refers to carbon-containing straight-chained, branch-chained and cyclic groups, all of which can be optionally substituted, containing at least one O, N or S heteroatom. The term “alkoxy” refers to the ether —O-alkyl, where alkyl is defined as above.

“Alkenyl” refers to unsaturated groups which contain at least one carbon-carbon double bond and includes straight-chain, branched-chain, and cyclic groups, all of which can be optionally substituted. P referable alkenyl groups have 1 to 10 carbon atoms. The term “heteroalkenyl” refers to unsaturated groups which contain at least one carbon-carbon double bond and includes straight-chained, branch-chained and cyclic groups, all of which can be optionally substituted, containing at least one O, N or S heteroatom.

“Anion” refers to an atom, molecule, or group of molecules having a net negative electrical charge.

“Aryl” refers to aromatic groups that have at least one ring having a conjugated, pi-electron system and includes carbocyclic aryl and biaryl, both of which can be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above.

“Central nervous system” (also written “CNS”) refers to the part of the nervous system that includes the brain and spinal cord. The central nervous system does not include the peripheral nerves which carry signals between the central nervous system and the muscles and organs of the body.

“Derivative” refers to a compound that is modified or partially substituted with another component.

“Effective amount” refers to an amount of an AChE, BChE, or AChE and BChE reactivation oxime or oxime containing composition for treatment purposes such that AChE, BChE, or AChE and BChE enzymes are reactivated in a therapeutically meaningful outcome. Determining an effective amount of such an oxime or combination of oximes for administering to a subject in need thereof can be done based on in vitro and/or animal data using routine computational methods well-known in the medical arts. A skilled person in the medical arts can determine what amount is sufficient for a therapeutically meaningful outcome. In one embodiment, the effective amount contains between about 200 g and 0.1 mg of one or more of the disclosed oximes. In another embodiment, the effective amount contains between about 100 g and 500 mg of one or more of the disclosed oximes. In a further embodiment, the effective amount contains between about 50 g and 1 g of one or more of the disclosed oximes, and preferably about 1-5 g thereof. A person of skill in the art will understand that the effective amount will depend on the mass of the subject and the extent of the exposure to the OP. In a still further embodiment, atropine is co-administered with the one or more of the disclosed oximes.

“Halo” refers to fluoro-, chloro-, bromo-, or iodo-substitutions. The term “alkanoyl” refers to the group. —C(O)R, where R is alkyl.

“Hydrocarbyl” refers to a hydrocarbon chain, which can be optionally substituted or provided with other substitutions known to the art.

“Organophosphorus compounds” (also written as “OP” or “OPs”) refer to esters of phosphoric acid which act on the enzyme acetylcholinesterase and have neurotoxicity. Such compounds include nerve agents such as tabun (Ethyl N,N-dimethylphosphoramidocyanidate, also referred to as GA), sarin (O-Isopropyl methylphosphonofluoridate, also referred to as GB), soman (O-Pinacolyl methylphosphonofluoridate, also referred to as GD), and VX (O-ethyl-S-[2(diisopropylamino)ethyl]methylphosphonothiolate), as well as some compounds used as insecticides, such as phosphoric acid diethyl 4-nitrophenyl ester (paraoxon), diethyl-p-nitrophenyl monothiophosphate (parathion) and phosphorothioic acid O-(3-chloro-4-methyl-2-oxo-2H-1-benzopyran-7-yl) O,O-diethyl ester (coumaphos).

“Pharmaceutical carriers” are well known in the medical art and include, but are not limited to, 0.01-0.1 molar phosphate buffer, 0.8% saline solution, propylene glycol, polyethylene glycol, vegetable oils and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishing agents, electrolyte replenishing agents such as those based on Ringer's dextrose, and the like. Preservatives and other additives may also be present, such as, for example, antimicrobials, antioxidants, chelating agents, inert gases and the like. Inert ingredients may further be used as an additive to a therapeutic compound or composition according to the present disclosure.

“Pharmaceutically acceptable” refers to any element, compound, or other molecule which does not interfere with the effectiveness of the biological activity of the present compounds and that is not toxic to the subject to which it is administered.

A “subject” refers a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs (“GP”), and the like). A “subject in need” refers to a subject that is at risk for exposure to OPs or that in need of a medical assistance to treat, reverse, counteract, and/or prevent poisoning, damage, and/or other harmful effects of exposure to OPs, whether intentional or accidental.

“Treat” and “treatment,” with respect to the exposure of a subject to an organophosphorus compound, refer to a medical intervention which attenuates, prevents, and/or counteracts the effects of such exposure. The foregoing terms can refer to the prophylactic administration of the present compounds and compositions, preferably in the form of a therapeutic composition comprising one or more of the disclosed oximes and one or more pharmaceutical carriers, to a subject at risk of exposure to an organophosphorus compound prior to an anticipated exposure, and/or can refer to the administration of the present compounds and compositions following such exposure.

Our laboratory has synthesized a series of phenoxyalkyl pyridinium oximes (see U.S. Pat. No. 9,227,937 B2 to Chambers et al. (the “'937 patent”), incorporated herein by reference in its entirety to the extent not inconsistent with the present application) which have shown the ability to reactivate acetylcholinesterase (AChE) inhibited by several OPs, and efficacy in preventing lethality from several OPs and entering the brain and attenuating seizure-like behavior (Chambers et al., 2016). The current line of investigation used several of these oximes to determine their ability to reactivate inhibited serum BChE by nerve agent surrogates PIMP (phthalimidyl isopropyl methylphosphonate, sarin surrogate), NEMP (nitrophenyl ethyl methylphosphonate, VX surrogate), NCMP (nitrophenyl cyclohexyl methyl phosphonate, cyclosarin surrogate), DFP (diisopropyl fluorophosphates, a common surrogate used in nerve agent testing), and two insecticidal OPs: paraoxon and phorate oxon, which are metabolites of the insecticides parathion and phorate, respectively. We have shown that the oximes tested (see TABLE 1) and the general class of compounds of use with inventive methods (see TABLE 2) are sufficiently able to penetrate the blood-brain barrier and are, therefore, effective as soon as they enter the brain, thereby producing a more effective antidote to OP compound poisoning. The present oximes, therefore, have increased lipophilicity, in order to enhance the ability of such molecules to pass into the brain. Lipophilicity can be determined experimentally by several methods, one of which is the octanol-water/buffer partition coefficient (Gulyaeva et al., 2003), a well-accepted, relatively easy method suitable for rapid screening of compounds. By targeting BChE, which is present in the circulating fluids of mammals, for reactivation, crossing the blood-brain barrier is not as great an issue. However, oxime molecules possessing dual reactivating properties (as shown herein) provide for an improved antidote/treatment compound by reactivating AChE in the CNS and BChE in the circulatory system and other fluid where it may be found. By reactivating BChE, this molecule can scavenge and destroy circulating and otherwise available OPs that potentially could inhibit AChE and cause poisoning of a subject.

Using the substituted phenoxyalkyl pyridinium oxime platform described in the '937 patent, the objective herein, therefore, is to identify some of these oxime molecules which can also reactivate the enzyme BChE. BChE is an esterase in the same family as AChE, which is the target enzyme of the OP anticholinesterases that leads to toxicity by these poisons. However, BChE inhibition does not lead to toxicity. BChE occurs in high levels in the serum of humans. At present, it can afford some protection to humans by being inhibited by the OPs instead of AChE; this inhibition is stoichiometric and destroys one molecule of OP for every BChE molecule that is inhibited. This BChE inhibition is persistent; therefore, the protection afforded is limited by the number of BChE molecules present in the serum. When BChE is inhibited by the OP in the serum, the enzyme and the OP are destroyed in a saturable 1:1 fashion, thus allowing significant amounts of OP to reach AChE.

We set out to determine if the inhibited BChE can be reactivated and the active site can be restored to its normal configuration, then it could be inhibited again, thus destroying another OP molecule, and a turnover situation could be established. Reactivating inhibited BChE could be viewed as a pseudo-catalytic mechanism of OP destruction and, therefore, the ability of BChE to protect from toxicity could be enhanced. Some of the novel oximes invented at Mississippi State University and listed in the '937 patent have now been shown by us in this disclosure to have the ability to reactivate BChE that has been inhibited by any of several OPs. Therefore, the potential exists for the development of this platform into BChE reactivators that could be antidotes for OP anticholinesterase toxicity by a mechanism not currently employed by existing antidotes. Ideally, the best chemicals identified herein display both activity as both AChE and BChE reactivators and are thus protective by displaying two therapeutic mechanisms.

As mentioned above, the novel phenoxyalkyl pyridinium oxime platform was invented by the Chambers laboratory at Mississippi State University with the purpose of providing improved therapy for the acute toxic effects of organophosphorus (OP) cholinesterase inhibitors such as nerve agents and/or insecticides. That research and development continues. The oximes currently being developed under the '937 patent are reactivators of the target enzyme AChE, and have the ability to counteract the toxic effects of OPs by restoring the activity of AChE not only in the peripheral nervous system but also in the brain and CNS. Some of the oximes in this platform are shown herein to also reactivate BChE, an enzyme in the blood that is inhibited by OPs but does not lead to toxicity in humans. Thus, BChE can provide some protection by destroying an OP molecule as it becomes inhibited, but this inhibition is persistent. The identified reactivating oximes will restore the BChE activity allowing it to destroy multiple OP molecules and reduce the OP concentration in the blood, reducing the likelihood of the OP molecules reaching the acute toxicity target AChE in the central and peripheral nervous systems. Therefore, these oximes have the potential to convert blood BChE into a pseudo-catalytic destruction mechanism and can provide therapeutic benefit. When these BChE reactivating oximes are concurrently AChE reactivators, they display two therapeutic mechanisms and are a substantial improvement over currently-approved antidotes.

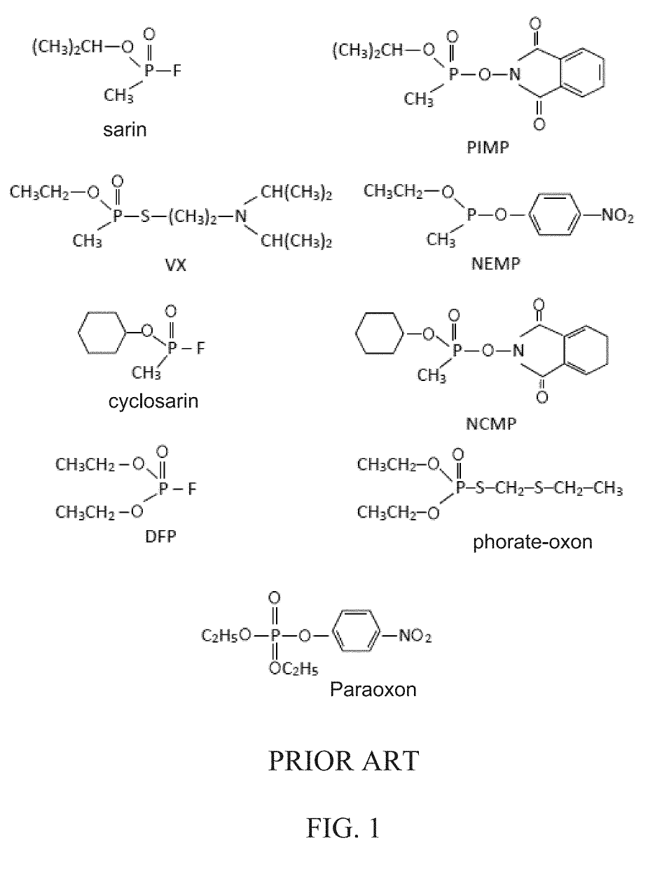

Now referring to FIG. 1, a panel of known (PRIOR ART) OP nerve agents, surrogates, and insecticidal oxons is shown. The specific oxime molecules tested in the present study for BChE reactivation are listed in TABLE 1. The tested oxime molecules are phenoxyalkyl pyridinium oximes that all share the common formula (I):

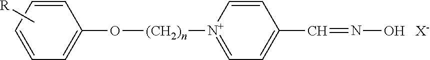

and include derivatives thereof. In these phenoxyalkyl pyridinium oxime compounds, “R” is hydrogen, alkyl, alkenyl, aryl, acyl, nitro, or halo; “n” is an integer selected from 3, 4, or 5; and “X”’ is a pharmaceutically acceptable anion. R can be, for example, a hydrogen, methyl, ethyl, phenyl, methoxy, ethoxy, trimethyl, methylchloro, diethyl, diethylchloro, ethylchloro, phenoxy, acetyl, benzoyl, bromo, chloro, iodo, dichloro, or trichloro substituent, or a combination of any of the foregoing. X⁻is preferably a halo moiety, such as a chlorine or bromine ion. Where appropriate, other pharmaceutically acceptable anions may be used. As shown in TABLE 1, the tested oxime molecules may differ in the linker alkyl chain length (n) as the number of carbon atoms in the chain and/or the phenoxy ring substitution moiety (R). For example, Oxime 14 (OX14) has a “n” value of “4” and a “R” value of “4-Cl—”, while Oxime 12 (OX12) has a “n” value of “5” and a “R” value of “4-CH₃—O—”. A list of phenoxyalkyl pyridinium oxime compounds that share characteristics with the tested oxime molecules is provided in TABLE 2. These molecules, may also be useful in the inventive methods of the present invention. For further information on chemical identity and synthesis the of phenoxyalkyl pyridinium oxime compounds, see the '937 patent.

TABLE 1
BChE Reactivation Tested Oxime Molecules

| Tested Phenoxyalkyl | alkyl linker | |
|---|---|---|
| Pyridinium Oxime | chain length | phenoxy substitution |
| Molecule | flu | moiety (R) |
| Oxime 12 (OX12) | 5 | 4-CH₃—O— |
| Oxime 14 (OX14) | 4 | 4-Cl— |
| Oxime 28 (OX28) | 4 | 4-CH₃CH₂C(:O)— |
| Oxime 31 (OX31) | 3 | 3-CH═CHCH═CH-4 |
| Oxime 32 (OX32) | 4 | 3-CH═CHCH═CH-4 |
| Oxime 59 (OX59) | 4 | 4-Ph—CH₂—O— |
| Oxime 98 (OX98) | 4 | 4-(CH₃)₃CCH₂C(CH₃)₂— |
| Oxime 99 (OX99) | 5 | 4-(CH₃)₃CCH₂C(CH₃)₂— |

TABLE 2
Phenoxyalkyl Pyridinium Oximes

| Oxime No. | R | n |
|---|---|---|
| 1, 2, 3 | H— | 3, 4, 5 |
| 4, 5, 6 | 4-CH₃— | 3, 4, 5 |
| 7, 8, 9 | 2,6-([CH₃]₂CH)₂— | 3, 4, 5 |
| 10, 11, 12 | 4-CH₃—O | 3, 4, 5 |
| 13, 14, 15 | 4-Cl— | 3, 4, 5 |
| 16, 17, 18 | 4-Br— | 3, 4, 5 |
| 19, 20, 21 | 4-O₂N— | 3, 4, 5 |
| 22, 23, 24 | 3-O₂N— | 3, 4, 5 |
| 25, 26, 27 | 4-CH₃C(:O)— | 3, 4, 5 |
| 28, 29, 30 | 4-CH₃CH₂C(:O)— | 3, 4, 5 |
| 31, 32, 33 | 3-CH═CHCH═CH-4 | 3, 4, 5 |
| 34, 35, 36 | 4-Ph | 3, 4, 5 |
| 37, 38, 39 | 2,3,5-(CH₃)₃— | 3, 4, 5 |
| 40, 41, 42 | 2,4,6-(CH₃)₃— | 3, 4, 5 |
| 43, 44, 45 | 3-CH₃-4-Cl— | 3, 4, 5 |
| 46, 47, 48 | 4-Ph—C(:O)— | 3, 4, 5 |
| 49, 50, 51 | 2,5-Cl₂— | 3, 4, 5 |
| 52, 53, 54 | 4-Ph—O— | 3, 4, 5 |
| 55, 56, 57 | 4-Ph—CH₂— | 3, 4, 5 |
| 58, 59, 60 | 4-Ph—CH₂—O— | 3, 4, 5 |
| 61, 62, 63 | 2,4,5-Cl₃— | 3, 4, 5 |
| 64, 65, 66 | 4-Ph—CH₂C(:O)— | 3, 4, 5 |
| 67, 68, 69 | 2,4,6-Cl₃— | 3, 4, 5 |
| 70, 71, 72 | 3,4-Cl₂— | 3, 4, 5 |
| 73, 74, 75 | 2,6-Cl₂-4-O₂N— | 3, 4, 5 |
| 76, 77, 78 | 4-Cl-3,5-(CH₃)₂— | 3, 4, 5 |
| 79, 80, 81 | 3-Ph— | 3, 4, 5 |
| 82, 83, 84 | 3-CH₃CH₂-4-Cl— | 3, 4, 5 |
| 85, 86, 87 | 3-O—C(:O)—CH═C(CH₃)-4 | 3, 4, 5 |
| 88, 89, 90 | 2-CH₃-4-(CH₃)₃C— | 3, 4, 5 |
| 91, 92, 93 | 2,4-[(CH₃)₃C—]₂— | 3, 4, 5 |
| 94, 95, 96 | 4-CH₃CH₂C(CH₃)₂— | 3, 4, 5 |
| 97, 98, 99 | 4-(CH₃)₃CCH₂C(CH₃)₂— | 3, 4, 5 |
| 100, 101, 102 | 2-Br-4-Cl— | 3, 4, 5 |
| 103, 104, 105 | 2-Cl-4-Br— | 3, 4, 5 |
| 106, 107, 108 | 2-Br-4-CH₃— | 3, 4, 5 |
| 109, 110, 111 | 4-Br-3,5-(CH₃)₂— | 3, 4, 5 |
| 112, 113, 114 | 4-CH₃(CH₂)₆—O— | 3, 4, 5 |
| 115, 116, 117 | 4-Ph—C(CH₃)₂— | 3, 4, 5 |
| 118, 119, 120 | 4-CH₃—O—CH₂CH₂— | 3, 4, 5 |
| 121, 122, 123 | 2,4-Cl₂— | 3, 4, 5 |

Methods: Butyrylcholinesterase activity was monitored using a discontinuous spectrophotometric assay with butyrylthiocholine as substrate and 5,5′-dithio-bis(nitrobenzoic acid) (DTNB) as chromogen after using a modification of the Ellman method (Chambers et al., 1988, Ellman et al., 1961). OP (in ethanol) or ethanol vehicle was added to serum (human, GP, or rat) in shaking water bath (37 ° C.) and incubated for 15 min to allow for an inhibition yield of about 80% in an Inhibition Phase. OP concentrations used were 3.16 μM for PIMP, 1.78 μM for NEMP, 1.78 μM for paraoxon, 10 μM for phorate oxon, 178 nM for DFP, and 3.16 μM for NCMP for an initial study with rat serum. A summary of the OP concentrations to achieve 80% BChE inhibition in rat, GP, and human serum is shown in TABLE 3. Oxime (100 μM; 1:1 DMSO:ETOH) was then added and incubated for 30 min in shaking water bath in a Reactivation Phase. Butyrylthiocholine (1 mM, final concentration) was added to all tubes and incubated for 15 minutes in a Substrate Hydrolysis Phase. The reactions were terminated and color developed with 0.01 M 5% SDS/DTNB (4:1) solution in a Termination Phase. Absorbance was measured at 412 nm. Eserine sulfate (10 μM) was incubated with a subset of control samples to account for non-enzymatic hydrolysis. One tailed T-test and Paired T-test were used to compare and measure significant differences between novel oximes in reference to 2-PAM.

TABLE 3
OP Inhibitory Concentrations of at least 80% BChE (expressed in μM)

| MODEL | PIMP | NEMP | PXN | PHO | PHOsox | DFP | NCMP |
|---|---|---|---|---|---|---|---|
| Rat | 3.16 | 1.78 | 1.78 | 10.0 | 1.00 | 0.178 | 3.16 |
| Guinea | 1.78 | 1.0 | 0.178 | 5.60 | TBD | TBD | TBD |
| Pig | | | | | | | |
| Human | 0.316 | 3.16 | 0.032 | 0.178 | TBD | TBD | TBD |

The phenoxyalkyl pyridinium oxime molecules and methods of the present invention provide the military with a more effective antidote against poisoning from nerve agents in the form of drugs available on a large scale. Moreover, civilians and the public in general would benefit through protection from and treatment for terrorist nerve agents and/or OP insecticides. As a result, the inventive methods provide for protection from and treatment of poisoning in many different scenarios.

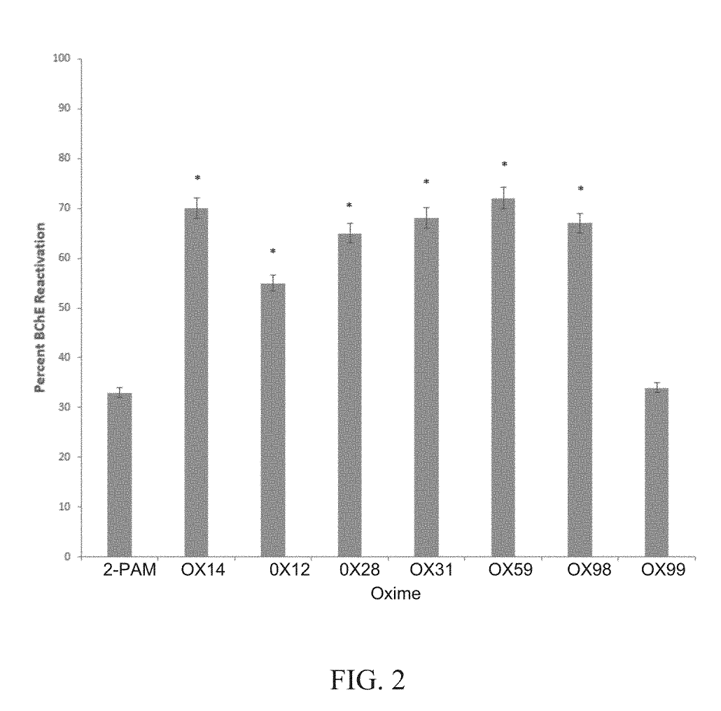

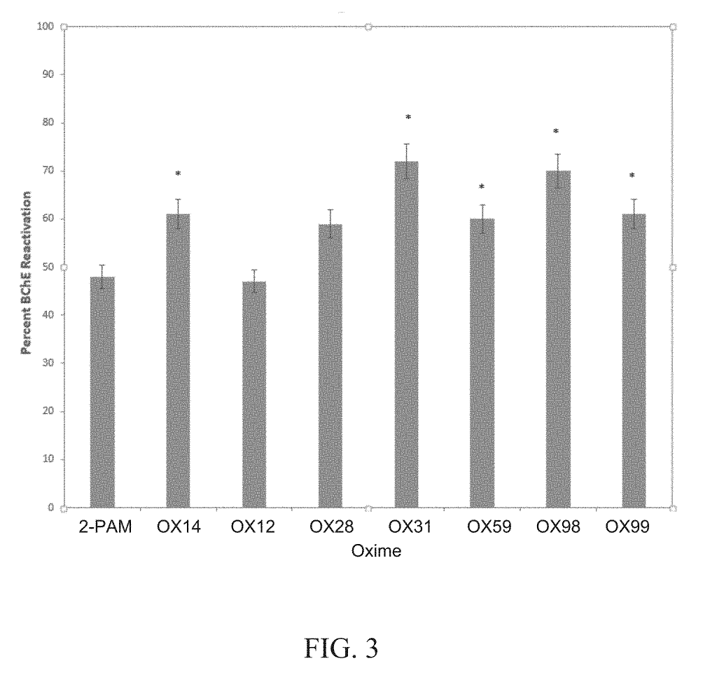

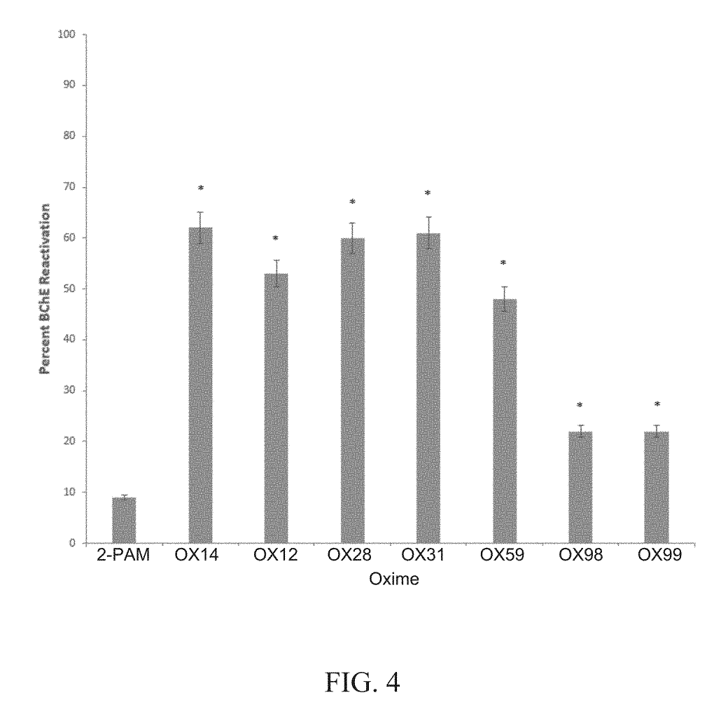

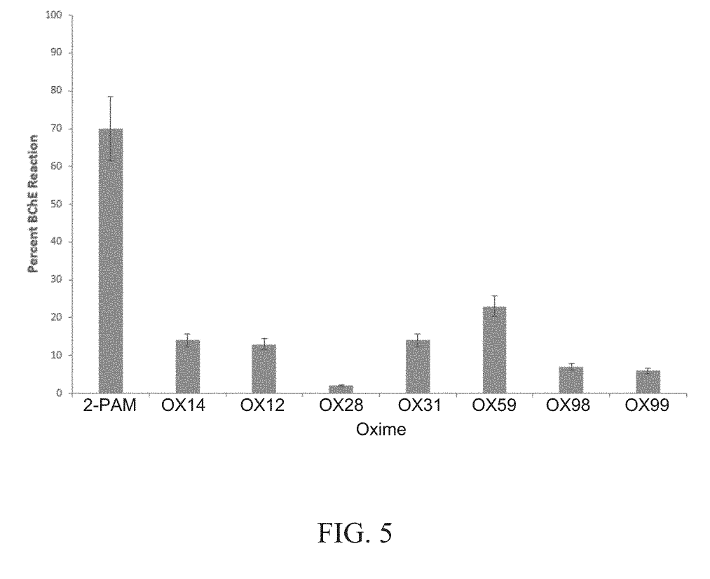

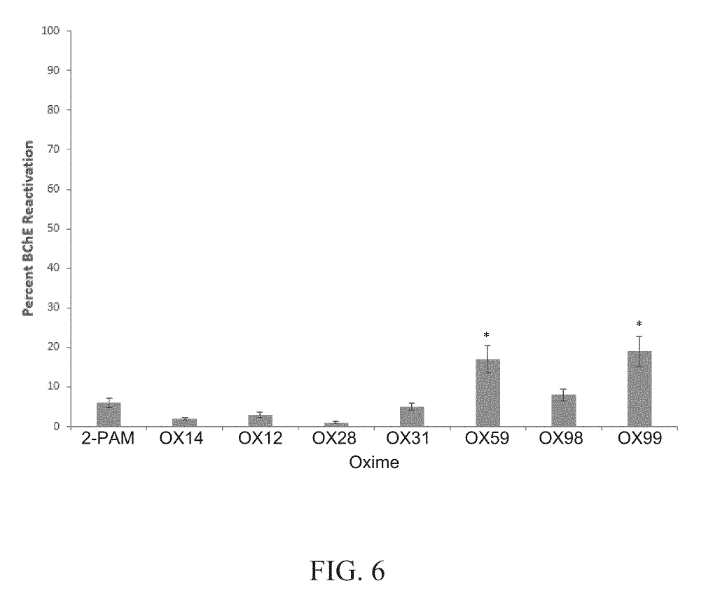

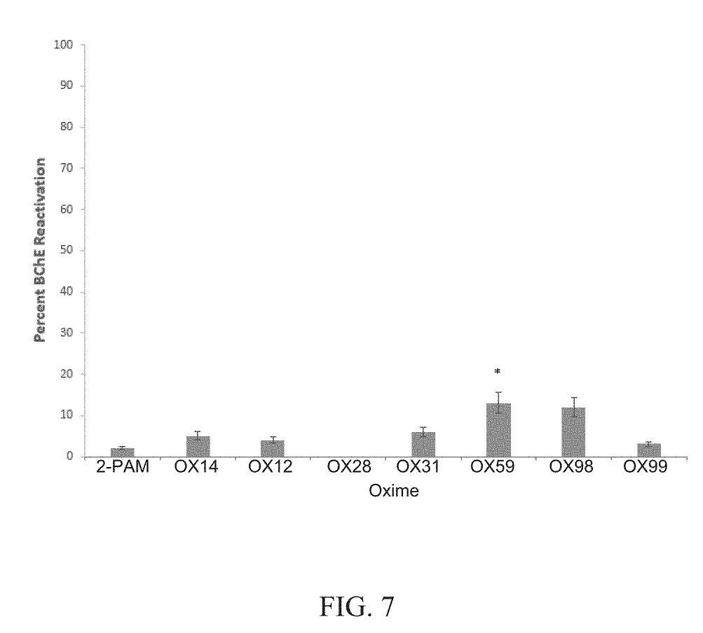

Referring now to FIGS. 2-7, rat serum BChE reactivation following exposure to various OPs with inhibition of at least 80% BChE. In FIG. 2, novel oximes (OX12, OX14, OX28, OX31, OX59, OX98, and OX99) exhibited a reactivation of BChE in a range of 33-72% after exposure to the insecticide paraoxon. The conventional antidote 2-PAM averaged 32% reactivation of BChE. All novel oximes except OX99 showed significant improvement of BChE reactivation over 2-PAM. In FIG. 3, novel oximes (OX12, OX14, OX28, OX31, OX59, OX98, and OX99) exhibited a reactivation of BChE in a range of 45-73% after exposure to sarin surrogate PIMP. The conventional antidote 2-PAM averaged 46% reactivation of BChE. All novel oximes except OX12 and OX28 showed significant improvement of BChE reactivation over 2-PAM. In FIG. 4, novel oximes (OX12, OX14, OX28, OX31, OX59, OX98, and OX99) exhibited a reactivation of BChE in a range of 18-62% after exposure to VX surrogate NEMP. The conventional antidote 2-PAM averaged 8% reactivation of BChE. All novel oximes showed significant improvement of BChE reactivation over 2-PAM. In FIG. 5, novel oximes (OX12, OX14, OX28, OX31, OX59, OX98, and OX99) exhibited a reactivation of BChE in a range of 0-24% after exposure to the insecticide phorate oxon. The conventional antidote 2-PAM averaged 70% reactivation of BChE. In FIG. 6, novel oximes (OX12, OX14, OX28, OX31, OX59, OX98, and OX99) exhibited a reactivation of BChE in a range of 0-21% after exposure to cyclosarin surrogate NCMP. The conventional antidote 2-PAM averaged 4% reactivation of BChE. Novel oximes OX59 and OX99 showed significant improvement of BChE reactivation over 2-PAM. In FIG. 7, novel oximes (OX12, OX14, OX28, OX31, OX59, OX98, and OX99) exhibited a reactivation of BChE in a range of 0-12% after exposure to a common surrogate used in nerve agent testing, DFP. The conventional antidote 2-PAM averaged 2% reactivation of BChE. Novel oxime OX59 showed significant improvement of BChE reactivation over 2-PAM.

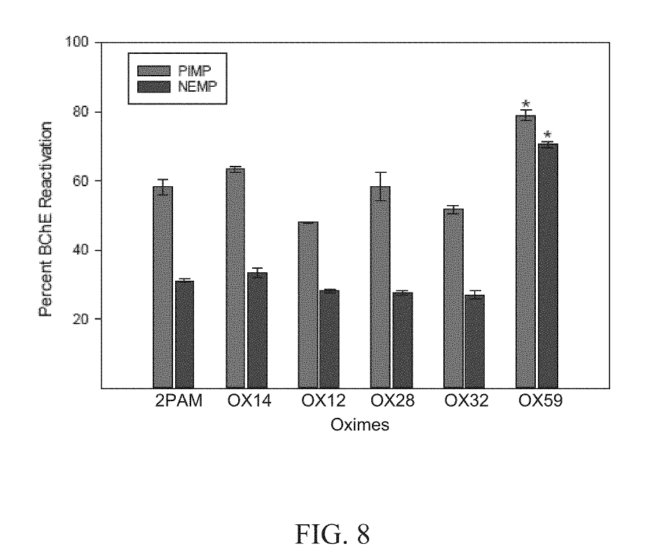

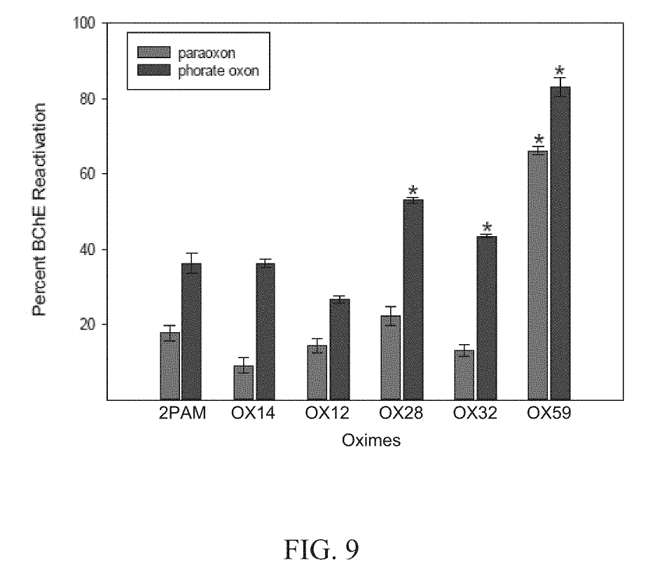

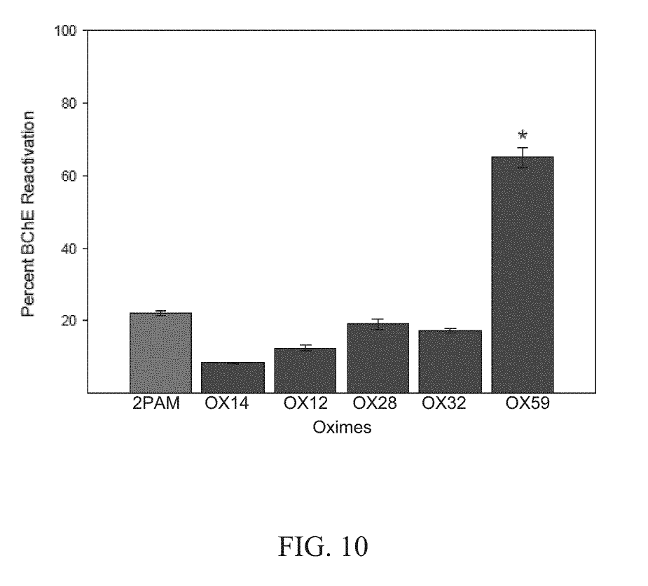

Referring now to FIGS. 8-16, in a second study, we conducted BChE reactivation experiments with three BChE serums (human, GP, and rat) in comparison to 2-PAM after exposure to various OPs with inhibition of at least 80% BChE. In FIG. 8, novel oximes (OX12, OX14, OX28, OX32, and OX59) exhibited a reactivation of human BChE in a range of 47-78% after exposure to PIMP and 27-70% to NEMP. The conventional antidote 2-PAM averaged 58% and 31% reactivation of BChE, respectively. Novel oxime OX59 showed significant improvement of BChE reactivation over 2-PAM for both PIMP and NEMP. In FIG. 9, novel oximes (OX12, OX14, OX28, OX32, and OX59) exhibited a reactivation of human BChE in a range of 9-66% after exposure to paraoxon and 26-83% to phorate oxon. The conventional antidote 2-PAM averaged 17% and 36% reactivation of BChE, respectively. Novel oxime OX59 showed significant improvement of BChE reactivation over 2-PAM for both paraoxon and phorate oxon, while novel oximes OX28 and OX32 showed significant improvement of BChE reactivation over 2-PAM for phorate oxon. In FIG. 10, novel oximes (OX12, OX14, OX28, OX32, and OX59) exhibited a reactivation of human BChE in a range of 8-65% after exposure to NCMP. The conventional antidote 2-PAM averaged 22% reactivation of BChE. Novel oxime OX59 showed significant improvement of BChE reactivation over 2-PAM for NCMP.

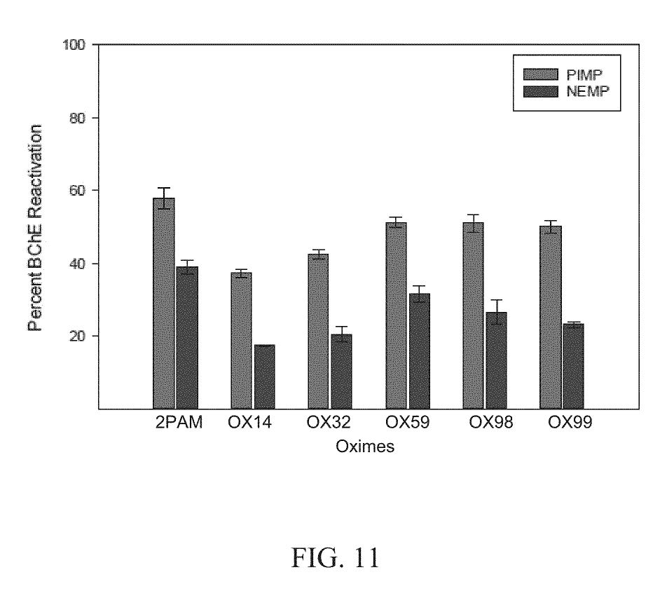

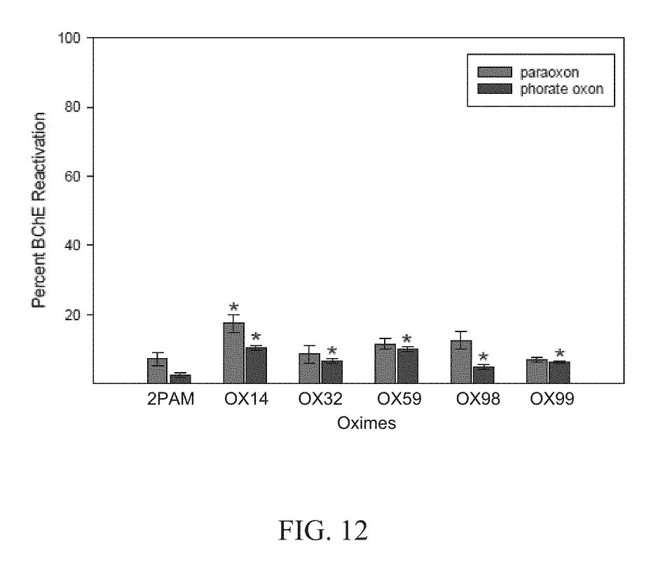

In FIG. 11, novel oximes (OX14, OX32, OX59, OX98, and OX99) exhibited a reactivation of GP BChE in a range of 42-51% after exposure to PIMP and 17-31% to NEMP. The conventional antidote 2-PAM averaged 57% and 38% reactivation of BChE, respectively. In FIG. 12, novel oximes (OX14, OX32, OX59, OX98, and OX99) exhibited a reactivation of GP BChE in a range of 6-17% after exposure to paraoxon and 4-10% to phorate oxon. The conventional antidote 2-PAM averaged 7% and 2% reactivation of BChE, respectively. Novel oxime OX14 showed significant improvement of BChE reactivation over 2-PAM for both paraoxon and phorate oxon, while novel oximes OX32, OX59, OX98, and OX99 showed significant improvement of BChE reactivation over 2-PAM for phorate oxon.

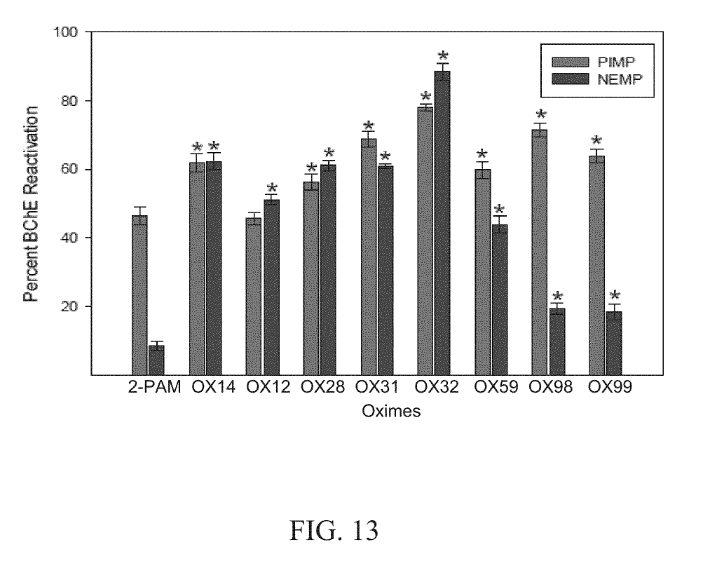

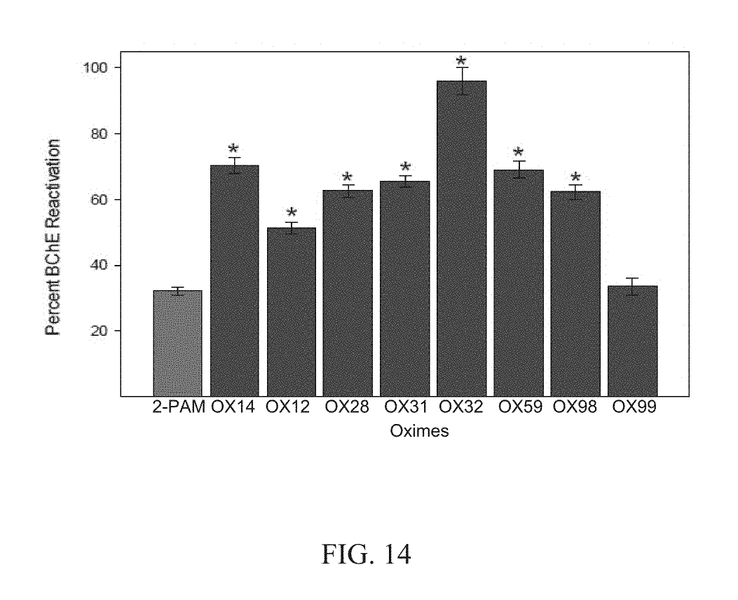

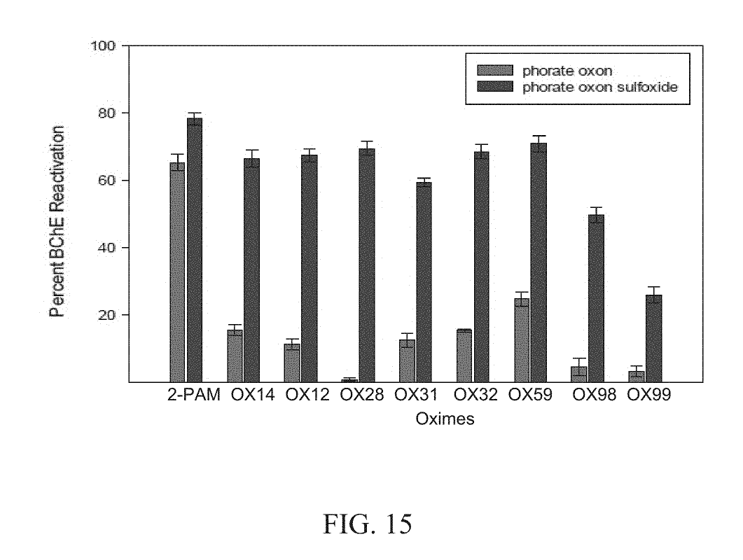

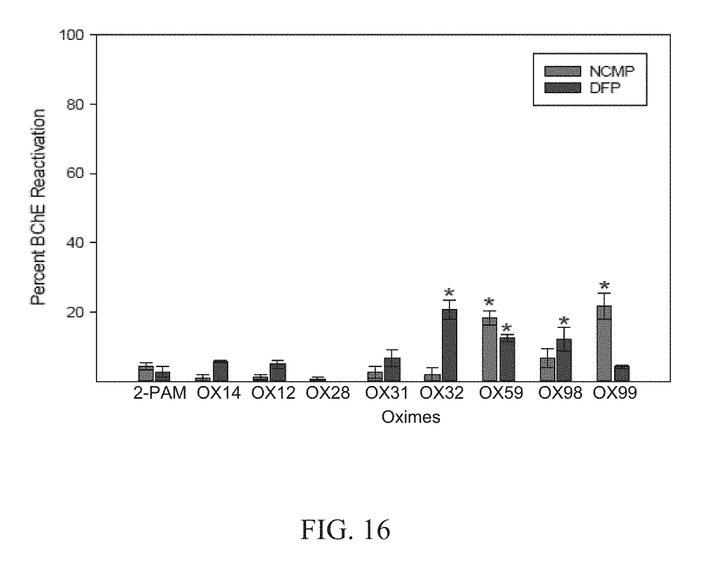

In FIG. 13, novel oximes (OX12, OX14, OX28, OX31, OX32, OX59, OX98, and OX99) exhibited a reactivation of rat BChE in a range of 45-77% after exposure to PIMP and 18-88% to NEMP. The conventional antidote 2-PAM averaged 57% and 38% reactivation of BChE, respectively. All novel oximes showed significant improvement of BChE reactivation over 2-PAM. In FIG. 14, novel oximes (OX12, OX14, OX28, OX31, OX32, OX59, OX98, and OX99) exhibited a reactivation of rat BChE in a range of 33-95% after exposure to paraoxon. The conventional antidote 2-PAM averaged 32% reactivation of BChE. All novel oximes except OX99 showed significant improvement of BChE reactivation over 2-PAM. In FIG. 15, novel oximes (OX14, OX32, OX59, OX98, and OX99) exhibited a reactivation of rat BChE in a range of 0-24% after exposure to phorate oxon and 25-70% to phorate oxon sulfoxide. The conventional antidote 2-PAM averaged 65% and 78% reactivation of BChE, respectively. In FIG. 16, novel oximes (OX12, OX14, OX28, OX31, OX59, OX98, and OX99) exhibited a reactivation of rat BChE in a range of 0-21% after exposure to NCMP and −20% to DFP. The conventional antidote 2-PAM averaged 4% and 2% reactivation of BChE, respectively. Novel oxime OX59 showed significant improvement of BChE reactivation over 2-PAM for both NCMP and DFP, while novel oximes OX99 showed significant improvement of BChE reactivation over 2-PAM for NCMP and novel oximes OX32 and OX98 showed significant improvement of BChE reactivation over 2-PAM for DFP.

Phorate oxon also has a thioester and a thioether; so size, charge, and bonding interactions could also interfere with the novel oxime-BChE reactivation capability. PIMP, NEMP, the sarin and VX surrogates, respectively, and paraoxon (the active metabolite of parathion), showed moderate to high reactivation of both AChE (see the '937 patent) and BChE with the oxime molecules tested (see FIGS. 2-4).

Phorate oxon, NCMP, and DFP showed little to no reactivation of BChE with the oxime molecules tested (see FIGS. 5-7). NCMP, a cyclosarin surrogate, and DFP have been traditionally very difficult to reactivate AChE. Both are very large molecules; NCMP has a cyclohexyl group, while DFP has two isopropyl groups, respectively, thus making access to the OP-BChE conjugate difficult for an oxime molecule. Reactivation after phorate oxon exposure showed more promise with AChE than BChE, which could suggest that our tested oxime molecules have better orientation and positioning within the active site of AChE than BChE after phorate oxon exposure.

The novel oximes tested in the second study group displayed different reactivation efficacies among human, guinea pig, and rat BChE (FIGS. 8-16). This could be due to differences in the size and structure of BChE among these species and the way our surrogates and oximes orient themselves in their respective active sites. Additionally, phorate oxon and phorate oxon sulfoxide, metabolites of phorate, displayed different potencies and reactivation efficacies. Novel oximes showed high reactivation efficacy in phorate oxon sulfoxide but not phorate oxon in rat BChE, despite the former being ten-fold more potent. Furthermore, paraoxon and the two phorate metabolites should theoretically display similar reactivation efficacies since both become diethyl phosphates after the leaving group departs, but showed substantial differences in reactivation, suggesting that the leaving group plays an important role in orientation of the OP in the active site. NCMP and DFP showed little to no reactivation in the rat and human, except OX59 in human BChE after inhibition by NCMP. These compounds have large R groups (cyclohexyl and diisopropyl, respectively) which could block the oxime from getting into the appropriate position to displace the phosphoryl group from the active site.

The nerve agent surrogates synthesized in our laboratory phosphorylate BChE and AChE serine hydrolases in a similar fashion as the actual nerve agents, suggesting our oximes may have similar efficacies, and thus making these studies relevant for new oxime therapeutics. Thus, administration of the novel oximes to a subject in need thereof represents a novel treatment mechanism by reactivating both AChE and BChE. The reactivation of BChE by the tested novel oximes also presents a new method of continually reactivating serum BChE with exposure of the BChE to the novel oximes in the circulation to act as a pseudo-catalyst for destroying OPs circulating in a subject's blood before the OP can inhibit a CNS AChE. We have shown significant broad spectrum capability with our novel oximes to reactivate both AChE (Chambers et al., 2013) and serum BChE in vitro after exposure to nerve agent and insecticidal OP chemistries, suggesting an alternative method in OP detoxication. OX59 showed at least 60% BChE reactivation in all human treatment groups, including NCMP (which is traditionally very difficult to reactivate), and showed high efficacy in the rat as well (statistically significant in all compounds). 2-PAM was a poor BChE reactivator overall, and all oximes were average/poor in guinea pig. Our oximes were more effective as potential pseudo-catalytic scavengers than 2-PAM, thus potentially affording more protection for AChE and mitigating toxic signs or lethality.

The results discussed above with BChE reactivation can be compared with the profile of AChE reactivation for 2-PAM and the tested oxime molecules in TABLE 4.

TABLE 4
In vitro AChE reactivation in rat brain homogenates by
2-PAM or novel oximes (data from Chambers, et al. (2013)).

| Oxime | Paraoxon | PIMP | NEMP | Phorateoxon | NCMP | DFP |
|---|---|---|---|---|---|---|
| 2-PAM | 87 | 91 | 88 | 73 | 6 | 25 |
| OX12 | 25 | 30 | 35 | 17 | 0 | 16 |
| OX14 | 56 | 54 | 52 | 56 | 0 | 23 |
| OX28 | 76 | 34 | 33 | 64 | 0 | 27 |
| OX31 | 45 | 65 | 57 | 26 | 0 | 12 |
| OX59 | 32 | 54 | 47 | 35 | 0 | 12 |
| OX98 | 93 | 51 | 56 | 79 | 4 | 50 |
| OX99 | 56 | 51 | 55 | 85 | 0 | 22 |

Because the nerve agent surrogates tested inhibit both butyrylcholinesterase and acetylcholinesterase with the same chemical moiety as the actual nerve agents, these reactivation studies are both timely and relevant. Several oxime molecules in our library have shown a significant ability to reactivate inhibited BChE in the serum by OPs, especially paraoxon and the sarin and VX surrogates, PIMP and NEMP, respectively. The more efficacious oxime molecules (OX14, OX28, OX31, and OX59) showed significant broad spectrum capabilities when compared to the current oxime reactivator 2-PAM. The ability to reactivate inhibited BChE could turn it into a pseudo-catalytic scavenger with appropriate dosing regimens of the efficacious oxime reactivators. Destruction of OP molecules by BChE in the serum could afford protection to AChE, or at the very least prevent severe poisoning against a diverse array of OPs. These novel oxime molecules can provide an alternative method in OP treatment.

### REFERENCES

Chambers, J. E., Meek E. C., Bennett J. P., Bennett W. S., Chambers H. W., Leach, C. A., Pringle, R. B., Wills R. W., (2016) Novel substituted phenoxyalkyl pyridinium oximes enhance survival and attenuate seizure-like behavior of rats receiving lethal levels of nerve agent surrogates. Toxicology, 339, 51-57.
Chambers, J. E., Wiygul, S. H., Harkness, J. E., and Chambers, H. W. (1988) Effects of acute paraoxon and atropine exposures on retention of shuttle avoidance behavior in rats. Neurosci. Res. Commun. 3, 85-92.
Chambers, J. E., Chambers, H. W., Pringle, R. B., (2013) Testing of novel brain-penetrating oxime reactivators of acetylcholinesterase inhibited by nerve agent surrogates. Chem Biol Interact, 1, 135-138.
Ellman, G. L., Courtney, K. D., Andres V., and Featherstone, R. M. (1961). A new and rapid colorimetric determination of acetylcholinesterase activity. Biochem. Pharmacol. 7, 88-95.
Meed, E. C., Chambers, H. W., Coban, A., Funck, K. E., Pringle, R. B., Ross, M. K., Chambers, J. E., (2012) Synthesis and in vitro and in vivo inhibition potencies of highly relevant nerve agent surrogates. Toxicol Sci, 2, 525-533.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A method of treating the effects of exposure to an organophosphorus compound in a subject in need thereof comprising administering an effective amount of a therapeutic composition comprising one or more phenoxyalkyl pyridinium oxime molecules and at least one pharmaceutical carrier to the subject, and reactivating serum butyrylcholinesterase in the subject.

2. The method of claim 1, wherein the one or more phenoxyalkyl pyridinium oxime molecules have the following formula: 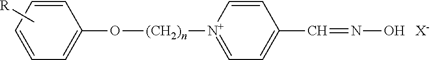
wherein: R is hydrogen, alkyl, alkenyl, aryl, alkoxy, aryloxy, acyl, nitro, or halo; n is 3, 4, or 5; and X⁻is a pharmaceutically acceptable anion; with the proviso that (1) when R is 4-CH₃CO—, n is not 5 and (2) when R is 4-Ph—CO—, n is neither 4 nor 5.

3. The method of claim 2, wherein the one or more phenoxyalkyl pyridinium oxime molecules are selected from the group consisting of
(a) n=5 and R=4-CH₃—O—;
(b) n=4 and R=4-Cl—;
(c) n=4 and R=4-CH₃CH₂C(:O)—;
(d) n=3 and R=3-CH═CHCH═CH-4;
(e) n=4 and R=4-Ph—CH₂—O—;
(f) n=4 and R=4-(CH₃)₃CCH₂C(CH₃)₂—; and
(g) n=5 and R=4-(CH₃)₃CCH₂C(CH₃)₂—.

4. The method of claim 2, wherein the one or more phenoxyalkyl pyridinium oxime molecules are selected from the group consisting of (1) n=4 and R=4-l—and (2) n=4 and R=4-Ph—CH₂—O—.

5. The method of claim 1 further comprising reactivating acetylcholinesterase in the subject.

6. The method of claim 1, wherein the reactivation of the serum butyrylcholinesterase is continual during exposure to the one or more phenoxyalkyl pyridinium oxime molecules such that the serum butyrylcholinesterase in the presence of the one or more phenoxyalkyl pyridinium oxime molecules acts as a pseudo-catalytic destruction mechanism of the organophosphorus compound.

7. The method of claim 1, wherein the administering step is effectuated by an administrative route selected from the group consisting of intravenous, oral, topical, intraperitoneal, transdermal, nasal, rectal, vaginal, intramuscular, and subcutaneous.

8. The method of claim 7, wherein the administering step is effectuated following exposure of the subject to the organophosphorus compound.

9. The method of claim 7, wherein the administering step is effectuated prior to exposure of the subject to the organophosphorus compound.

10. The method of claim 1, wherein the organophosphorus compound is an insecticide.

11. The method of claim 1, wherein the organophosphorus compound is sarin, soman, tabun, VX, or combinations thereof.

12. A method of preventing acetylcholinesterase poisoning in a subject in need thereof comprising administering an effective amount of a therapeutic composition comprising one or more phenoxyalkyl pyridinium oxime molecules and at least one pharmaceutical carrier to the subject, and reactivating serum butyrylcholinesterase in the subject.

13. The method of claim 12, wherein the reactivation of the serum butyrylcholinesterase is continual during exposure to the one or more phenoxyalkyl pyridinium oxime molecules such that the serum butyrylcholinesterase in the presence of the one or more phenoxyalkyl pyridinium oxime molecules acts as a pseudo-catalytic destruction mechanism of the organophosphorus compound.

14. The method of claim 13, wherein the one or more phenoxyalkyl pyridinium oxime molecules have the following formula: 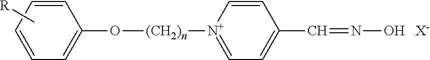
wherein: R is hydrogen, alkyl, alkenyl, aryl, alkoxy, aryloxy, acyl, nitro, or halo; n is 3, 4, or 5; and X⁻is a pharmaceutically acceptable anion; with the proviso that (1) when R is 4-CH₃CO—, n is not 5 and (2) when R is 4-Ph—CO—, n is neither 4 nor 5.

15. The method of claim 14, wherein the one or more phenoxyalkyl pyridinium oxime molecules are selected from the group consisting of
(a) n=5 and R=4-CH₃—O—;
(b) n=4 and R=4-Cl—;
(c) n=4 and R=4-CH₃CH₂C(:O)—;
(d) n=3 and R=3-CH═CHCH═CH-4;
(e) n=4 and R=4-Ph—CH₂—O—;
(f) n=4 and R=4-(CH₃)₃CCH₂C(CH₃)₂—; and
(g) n=5 and R=4-(CH₃)₃CCH₂C(CH₃)₂—.

16. The method of claim 15, wherein the one or more phenoxyalkyl pyridinium oxime molecules are selected from the group consisting of (1) n=4 and R=4-Cl—and (2) n=4 and R=4-Ph—CH₂—O—.

17. The method of claim 12 further comprising reactivating acetylcholinesterase in the subject.

18. A method of clearing of an organophosphorus compound within the circulatory system a subject in need thereof comprising administering an effective amount of a therapeutic composition comprising one or more phenoxyalkyl pyridinium oxime molecules and at least one pharmaceutical carrier to the subject, and reactivating serum butyrylcholinesterase in the subject.

19. The method of claim 18, wherein the reactivation of the serum butyrylcholinesterase is continual during exposure to the one or more phenoxyalkyl pyridinium oxime molecules such that the serum butyrylcholinesterase in the presence of the one or more phenoxyalkyl pyridinium oxime molecules acts as a pseudo-catalytic destruction mechanism of the organophosphorus compound.

20. The method of claim 19 further comprising reactivating acetylcholinesterase in the subject.
